(19) United States
(12) Patent Application Publication
(10) Pub. No.: US 2007/0111269 A1
(43) Pub. Date: May 17, 2007

(54) METHOD TO PREDICT POSITIVE REPEAT PROSTATE BIOPSY

(76) Inventors: Michael Kattan, Cleveland Heights, OH (US); Ernesto Lopez-Corona, Chula Vista, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/420,113

(22) Filed: May 24, 2006

Related U.S. Application Data
(63) Continuation of application No. PCT/US04/39456, filed on Nov. 24, 2004.
(60) Provisional application No. 60/525,323, filed on Nov. 26, 2003.

(51) Int. Cl. G01N 33/574; G06F 19/00

(52) U.S. Cl. 435/7.23; 702/19

(57) ABSTRACT

A method and system for prognosis of prostate cancer in a patient after a negative biopsy.

### CROSS-REFERENCE TO RELATED APPLICATIONS

This application is a continuation under 35 U.S.C. 111(a) of PCT/US2004/039456 filed on Nov. 24, 2004, and published in English on Jun. 16, 2005 as WO 2005/055115 A3, which claims the benefit of the filing date of U.S. application Ser. No. 60/525,323, filed Nov. 26, 2003 under 35 U.S.C. § 119(e), which publication and disclosure are incorporated by reference herein.

### BACKGROUND

Approximately 189,000 new cases of prostate cancer were detected in 2002 in the United States (Jemal et al., 2002) and, in the majority, high prostate specific antigen (PSA) was the main reason for pursuing the diagnosis. In 2004, the American Cancer Society estimates 230,110 men will be diagnosed with prostate cancer in the United States and predicts 29,900 deaths will be attributed to it (Cancer Facts & Figures, 2004). Although most cancer is found at the initial biopsy session, 70% to 80% of patients who undergo biopsy are left with doubt regarding the presence of prostate cancer. Because of this uncertainty and the false-negative rate of an initial biopsy session, currently an increased number of patient undergo a subsequent biopsy session (O'Dowd, 2000; Keetch et al., 1996).

In about 20% of patients, prostate cancer is detected at a second biopsy session and different markers or variables have been suggested to indicate a higher likelihood for finding cancer on repeat prostate biopsy (Keetch et al., 1994; Keetch et al., 1996; Fowler et al., 2000; Djavan et al., 2000; Epstein et al., 2001; Vis et al., 2000; Roehrborn et al., 1996; Ellis et al., 1995; Park et al., 2001; Raviv et al., 1996; Keetch et al., 1995; O'Dowd et al., 2000; Ouyang et al., 2001; Cheville et al., 1997). By selecting patients who have a specific marker, a subset can be identified with a 60% chance of cancer detection (Ellis et al., 1995; Fleshner et al., 1997). Of these markers, clinical and histological variables have been considered. The clinical variables generally studied are age, race, a suspicious lesion on digital rectal examination (DRE), total serum PSA, the ratio of free-to-total PSA, PSA slope or PSA velocity higher than 0.75 yearly, PSA density above 0.15 and transition zone density higher than 0.25. Of the pathological findings in previous biopsies, high grade intraepithelial neoplasia (HGPIN) and atypical small acinar proliferation (ASAP) are known to be associated with a higher likelihood of finding cancer in a subsequent biopsy (Keetch et al., 1994; Keetch et al., 1996; Fowler et al., 2000; Djavan et al., 2000; Epstein et al., 2001; Vis et al., 2000; Roehrborn et al., 1996). The major problem with these markers is that, while they permit the identification of a high risk subset, when used individually they do not help isolate a group of patients who are at low risk for prostate cancer. Consequently, a recommendation to withhold subsequent biopsy cannot be sustained. Although knowledge of the risk that one particular variable carries of finding cancer in a subsequent biopsy is useful for clinicopathological research and clinical trial design, patient counseling requires the integration of various prognostic factors to arrive at a single prediction for the individual. That is, simply counting patient risk factors does not optimize the information available enough to predict the finding of undiagnosed cancer and to decide, when the risk is sufficiently low, to stop performing additional biopsies.

What is needed is an improved method to predict a positive repeat prostate biopsy after a negative prostate biopsy.

### SUMMARY OF THE INVENTION

The invention provides methods, apparatus and nomograms to predict a positive repeat prostate biopsy, e.g., to predict the disease free status, after a negative biopsy in a patient at risk or suspected of having prostate cancer. The methods employ clinical and/or pathological values or scores for one or more factors such as age, DRE, PSA, e.g., percent free PSA, transition zone PSA density, BPSA, and/or proPSA, PSA slope, family history, history of HGPIN, history of ASAP, number of months since initial negative biopsy, number of months from previous negative biopsy, imaging data, biopsy data, such as data from at least a 6 core and optionally up to at least a 10, e.g., at least a 12 core biopsy set, and/or other markers, e.g., markers present in a physiological fluid sample, such as a protein found in the blood other than PSA, expression data, e.g., DD3/PCA3 expression, and/or genomic data, to predict a positive repeat prostate biopsy after a negative biopsy and/or the timing of a repeat biopsy, i.e., whether to repeat a biopsy or wait. In one embodiment of the invention, the marker in the physiological fluid sample includes one or more of TGF-beta, UPA, UPAR, PAI-1, IL-6, IL6sR, IGF BP-2, IGF BP-3, p53, Ki-67, p21, E-cadherin, as well as VEGF, and VCAM, e.g., sVCAM. As used herein, a sample of “physiological body fluid” includes, but is not limited to, a sample of blood, plasma, serum, seminal fluid, urine, saliva, sputum, semen, pleural effusions, bladder washes, bronchioalveolar lavages, cerebrospinal fluid and the like. Thus, patient specific factors including the presence or amount of markers in physiological fluid, such as PSA, are useful to predict a positive repeat prostate biopsy in a patient after a negative biopsy.

As described herein, 343 patients were studied with at least one initial negative biopsy. At each biopsy session, patient age, family history of prostate cancer, serum prostate specific antigen (PSA), PSA slope, digital rectal examination findings, the number of months from the initial biopsy session, the cumulative number of negative cores previously obtained, and the history of high grade prostatic intraepithelial neoplasia (HGPIN) or atypical small acinar proliferation (ASAP) were recorded. Through Cox regression analysis, the association of each variable with time to a positive biopsy was determined. A nomogram was constructed using all variables and discrimination was calculated as the concordance index. There were 661 biopsy sessions. A mean of 2.9 biopsy sessions were performed per patient and a mean of 9.15 cores were obtained per biopsy session for a mean of 25.2 per patient. Overall 20% of patients had cancer at the second biopsy session. The cumulative number of negative cores obtained, PSA slope, history of HGPIN, and history of ASAP were associated with repeat biopsy findings (all p<0.05). Logistic regression analysis was performed to assess the relationships of individual variables with time to positive repeat biopsy. A nomogram was constructed that had a concordance index of 0.70, which was greater than that of any single risk factor. The nomogram predicts a positive biopsy after a previous negative biopsy session and whether a repeat biopsy is indicated, i.e., should be performed at the present time. The nomogram can thus improve clinical judgment before the decision to repeat biopsy.

As also described herein, 356 repeat biopsies from 230 patients for a mean of 2.56 total biopsies per patient were evaluated to assess the validity of the nomogram applied to an external dataset. The mean number of total cores per patient was 17.9. There were 78 positive biopsies. The predictor variables studied in the nomogram included patient age, family history of prostate cancer, digital rectal examination, serum PSA, PSA slope, months from initial negative biopsy session, months from previous negative biopsy session, cumulative number of negative cores previously taken, and history of HGPIN or ASAP. The nomogram predicted probability was calculated for each patient and those predicted outcomes were compared with the actual biopsy results. Area under the ROC curve was calculated as a measure of discrimination. Calibration was assessed graphically. The area under the ROC curve was 0.71 which was greater than any single risk factor. Calibration of the nomogram appeared to be very good. Therefore, the nomogram can provide important additional information to aid the urologist and patient with a negative biopsy in evaluating clinical options.

Accordingly, certain values or scores from a male at risk of or suspected of having prostate cancer are prognostically useful, and may optionally be employed in conjunction with other information including other clinical and/or pathological factors. In one embodiment, the prognosis is based on a computer derived analysis of data of the amount, level or other value (score) for one or more factors. Data may be input manually or obtained automatically from an apparatus.

In one embodiment, the invention includes correlating various clinical and/or pathological values or scores, for example, in a nomogram, to predict a positive repeat prostate biopsy, e.g., the presence of prostate cancer, after negative biopsy, and/or the timing of a repeat biopsy for a patient at risk of or suspected of having prostate cancer. In one embodiment, the method employs factors such as PSA slope, number of months from a previous negative biopsy, and age, and/or optionally number of months from an initial negative biopsy, number of previous negative cores, DRE, PSA, history of HGPIN and/or history of ASAP. In one embodiment, the concordance index is at least 0.70 when two or more factors are employed.

In one embodiment, the invention provides a method to determine a probability of a positive repeat prostate biopsy after a negative biopsy in a patient suspected of having prostate cancer. The method includes inputting test information to a data input means, wherein the information includes one or more, e.g., two, three, four, five, six, seven, eight, nine, ten or more, patient factors including age, DRE, PSA, PSA slope, number of negative cores, family history of prostate cancer, history of HGPIN, history of ASAP, number of months since initial negative biopsy, or numbers of months from previous negative biopsy, executing a software for analysis of the test information, and analyzing the test information so as to provide a probability of a positive repeat prostate biopsy after a negative biopsy in the patient.

Further provided is a method to determine whether or when to repeat a prostate biopsy after a negative biopsy in a patient suspected of having prostate cancer. The method includes inputting test information to a data input means, wherein the information includes one or more patient factors including age, DRE, PSA, PSA slope, number of negative cores, family history of prostate cancer, history of HGPIN, history of ASAP, number of months since initial negative biopsy, or number of months from previous negative biopsy, executing a software for analysis of the test information, and analyzing the test information so as to determine whether or when to repeat a prostate biopsy, i.e., determine the need for a repeat biopsy in the patient.

The invention also provides method to predict a probability of a positive repeat prostate biopsy after a negative biopsy in a patient suspected of having prostate cancer. The method includes correlating one or more factors for a patient to a functional representation of one or more factors determined for each of a plurality of persons previously subjected to two or more prostate biopsies, at least the first of which was negative and one of which was positive so as to yield a value for total points for the patient. Factors for each of a plurality of persons are correlated with the probability of a positive repeat prostate biopsy after a negative biopsy for each person in the plurality of persons, wherein the one or more factors include age, DRE results, PSA level, PSA slope, number of negative cores, family history, history of ASAP, history of HGPIN, number of months since previous negative biopsy and/or number of months from an initial negative biopsy. The functional representation includes a scale for each of age, DRE, PSA, PSA slope, number of negative cores, family history, history of HGPIN, history of ASAP, number of months since initial negative biopsy, and/or number of months from previous negative biopsy, a points scale, a total points scale, and a predictor scale. The scales for age, DRE, PSA, PSA slope, number of negative cores, family history, history of HGPIN, history of ASAP, number of months since initial negative biopsy, and/or number of months from previous negative biopsy, each have values on the scales which can be correlated with values on the points scale, and the total points scale has values which may be correlated with values on the predictor scale. The value on the total points scale for the patient is correlated with a value on the predictor scale to predict the quantitative probability of a positive repeat prostate biopsy after a negative biopsy in the patient.

Also provided is a method to determine whether to repeat a prostate biopsy after a negative biopsy in a patient suspected of having prostate cancer. The method includes correlating one or more factors for a patient to a functional representation of one or more factors determined for each of a plurality of persons previously subjected to two or more prostate biopsies, at least the first of which was negative and one of which was positive so as to yield a value for total points for the patient. The factors for each of a plurality of persons are correlated with a determination of whether or when to repeat a prostate biopsy after a negative biopsy for each person in the plurality of persons. The one or more factors include age, PSA level, PSA slope, number of negative cores, family history, DRE results, history of ASAP, history of HGPIN, number of months since previous negative biopsy and/or number of months from an initial negative biopsy, wherein the functional representation includes a scale for each of age, DRE, PSA, PSA slope, number of negative cores, family history, history of HGPIN, history of ASAP, number of months since initial negative biopsy, and/or number of months from previous negative biopsy, a points scale, a total points scale, and a predictor scale. The scales for age, DRE, PSA, PSA slope, number of negative cores, family history, history of HGPIN, history of ASAP, number of months since initial negative biopsy, and/or number of months from previous negative biopsy, each have values on the scales which can be correlated with values on the points scale, and the total points scale has values which may be correlated with values on the predictor scale. The value on the total points scale for the patient is correlated with a value on the predictor scale to determine whether or when to repeat a prostate biopsy after a negative biopsy in a patient suspected of having prostate cancer.

Thus, nomograms incorporating a variety of variables can predict a probability that a male at risk of or suspected of having prostate cancer has a positive repeat prostate biopsy after a negative biopsy or whether or when a repeat biopsy should be performed.

The invention also provides an apparatus. The apparatus includes data input means, for input of test information comprising the score, level or amount of at least one factor such as age, DRE, PSA, PSA slope, number of negative cores, family history, history of HGPIN, history of ASAP, months since initial negative biopsy, and/or months from previous negative biopsy in a male at risk of or suspected of having prostate cancer, and a processor, executing a software for analysis of the score, level or amount of the at least one factor. The software analyzes the score, level or amount of the at least one factor and provides the probability of a positive repeat prostate biopsy after a negative biopsy and/or whether or not or when to repeat a prostate biopsy.

Further provided is an apparatus to predict a probability of a positive repeat prostate biopsy after a negative biopsy in a patient suspected of having prostate cancer. The apparatus includes a correlation of one or more factors for each of a plurality of persons subjected to at least two prostate biopsies, at least the first of which was negative and one of which was positive, with the probability of a positive repeat prostate biopsy after a negative biopsy for each person of the plurality of persons. The one or more factors include age, DRE, PSA, PSA slope, number of negative cores, family history, history of HGPIN, history of ASAP, the number of months since initial negative biopsy, and/or the number of months from previous negative biopsy. The apparatus also includes a means for comparing an identical one or more factors determined from the patient to the correlation to predict the quantitative probability of a positive repeat prostate biopsy in the patient.

In one embodiment, the invention provides an apparatus to determine whether or when a repeat prostate biopsy is indicated after a negative biopsy in a patient suspected of having prostate cancer. The apparatus includes a correlation of one or more factors for each of a plurality of persons subjected to at least two prostate biopsies, at least the first of which was negative and one of which was positive, with whether a repeat prostate biopsy is indicated after a negative biopsy for each person of the plurality of persons. The one or more factors include age, DRE, PSA, PSA slope, number of negative cores, family history, history of HGPIN, history of ASAP, number months since initial negative biopsy, and/or number of months from previous negative biopsy. The apparatus further includes a means for comparing an identical one or more factors determined from the patient to the correlation to determine whether or when a repeat prostate biopsy is indicated.

In one embodiment, the invention provides an apparatus for predicting a probability of a positive repeat prostate biopsy after a negative biopsy in a patient suspected of having prostate cancer. The apparatus includes a scale for one or more of age, DRE, PSA, PSA slope, number of negative cores, family history, history of HGPIN, history of ASAP, number of months since initial negative biopsy, and/or number of months from previous negative biopsy, a points scale, a total points scale and a predictor scale. The scales for one or more of age, DRE, PSA, PSA slope, number of negative cores, family history, history of HGPIN, history of ASAP, number of months since initial negative biopsy, and/or number months from previous negative biopsy, each has values on the scales, and the scales for age, DRE, PSA, PSA slope, number of negative cores, family history, history of HGPIN, history of ASAP, number of months since initial negative biopsy, and/or number of months from previous negative biopsy, are disposed so that each of the values on the age, DRE, PSA, PSA slope, number of negative cores, family history, history of HGPIN, history of ASAP, number of months since initial negative biopsy, and/or number of months from previous negative biopsy, can be correlated with values on the points scale. The total points scale has values on the total points scale and the total points scale is disposed on the solid support with respect to the predictor scale so that the values on the total points scale may be correlated with values on the predictor scale, such that the values on the points scale correlating with the patient's age, DRE, PSA, PSA slope, number of negative cores, family history, history of HGPIN, history of ASAP, number of months since initial negative biopsy, and/or number of months from previous negative biopsy, can be added together to yield a total points value. The total points value can then be correlated with the predictor scale to predict a positive repeat prostate biopsy after a negative biopsy.

An apparatus is further provided for predicting whether or when to repeat a prostate biopsy after a negative biopsy in a patient suspected of having prostate cancer. The apparatus includes a scale for one or more of age, DRE, PSA, PSA slope, number of negative cores, family history, history of HGPIN, history of ASAP, number of months since initial negative biopsy, and/or number of months from previous negative biopsy, a points scale, a total points scale and a predictor scale. The scales for one or more of age, DRE, PSA, PSA slope, number of negative cores, family history, history of HGPIN, history of ASAP, number of months since initial negative biopsy, and/or number months from previous negative biopsy, each has values on the scales, and the scales for age, DRE, PSA, PSA slope, number of negative cores, family history, history of HGPIN, history of ASAP, number of months since initial negative biopsy, and/or number of months from previous negative biopsy, are disposed so that each of the values on the age, DRE, PSA, PSA slope, number of negative cores, family history, history of HGPIN, history of ASAP, number of months since initial negative biopsy, and/or number of months from previous negative biopsy, can be correlated with values on the points scale. The total points scale has values on the total points scale, and scale is disposed on the solid support with respect to the predictor scale so that the values on the total points scale may be correlated with values on the predictor scale, such that the values on the points scale correlating with the patient's age, DRE, PSA, PSA slope, number of negative cores, family history, history of HGPIN, history of ASAP, number of months since initial negative biopsy, and/or number of months from previous negative biopsy, can be added together to yield a total points value, and the total points value can be correlated with the predictor scale to predict whether or when to repeat a prostate biopsy in the patient.

Also provided is a system. The system includes a processor, an input device, an output device, a storage device, a database, wherein the database includes data collected from a plurality of patients having more than one prostate biopsy at least the first of which was negative and one of which was positive, and software. The software is operable on the processor to receive input from the input device, the input including one or more factors for determining a probability of a positive repeat prostate biopsy after a negative biopsy in a patient suspected of having prostate cancer and/or whether to perform a repeat biopsy after a negative biopsy in a patient suspected of having prostate cancer; and correlate received input with the collected data from the plurality of patients having more than one prostate biopsy to determine a prognosis probability or perform a repeat biopsy.

In one embodiment, a system includes a processor, an input device, an output device, a storage device, a database wherein the database includes data collected from a plurality of patients having more than one prostate biopsy, at least the first of which was negative and one of which was positive, and software operable on the processor. The software operable on the processor receives input from the input device, the input including one or more factors for determining whether or when to repeat a prostate biopsy after a negative biopsy in a patient suspected of having prostate cancer, and correlates received input with the collected data from the plurality of patients to determine whether or when to perform a repeat biopsy in the patient.

A system for predicting a positive repeat prostate biopsy after a negative biopsy in a patient suspected of having prostate cancer is further provided. The system includes a data structure for storing historic prostate cancer data, the structure contained in a memory and including one or more factors each corresponding to a characteristic of prostate cancer, and a processing device including program means for correlating the one or more factors corresponding to characteristics of prostate cancer with factor data collected from a patient suspected of having prostate cancer, which patient has a negative prostate biopsy, wherein the correlating results in a probability of a positive repeat prostate biopsy output by the processing device.

Also provided is a system for predicting whether or when to repeat prostate biopsy after a negative biopsy in a patient suspected of having prostate cancer. The system includes a data structure for storing historic prostate cancer data, the structure contained in a memory and comprising one or more factors each corresponding to a characteristic of prostate cancer, and a processing device including program means for correlating the one or more factors corresponding to characteristics of prostate cancer with factor data collected from a patient suspected of having prostate cancer, which patient has a negative prostate biopsy, wherein the correlating results in a probability of prostate cancer which is indicative of whether or when to repeat a prostate biopsy, output by the processing device.

### BRIEF DESCRIPTION OF THE DRAWINGS

FIG. 1A. Repeat biopsy (Bx) nomogram based on 343 patients. Each scale position has corresponding prognostic points (top axis). Point values for all predictor variables are determined consecutively and summed to arrive at total point value. This value is plotted on total points axis and directly below it is prediction for finding cancer depending on whether the patient is presently 12, 24 or 36 months out from initial negative biopsy. For age use age at current biopsy. For DRE use current DRE result. For months (Mo.) from first biopsy use months from first biopsy to today. For negative (Neg.) cores previously (prev.) taken use cumulative cores previously removed. For history of HGPIN and ASAP use affirmative when they were found before at any biopsy session. For PSA use current serum PSA. For PSA slope subtract pervious serum PSA measurement from current PSA and divide this difference by years between PSA measurements. Family history is positive when first or second degree relative has been diagnosed with prostate cancer. N, negative. ≦½ lobe, suspicious nodule less than half lobe. >½ lobe, suspicious nodule greater than half lobe. Prob., probability. +, positive.

FIG. 1B. Additional repeat biopsy nomogram

FIG. 2. Nomogram predicted probability for positive repeat biopsy showing nomogram calibration. Ideal nomogram would have predicted probabilities that match actual probabilities, i.e., lie on 45-degree solid line. Predictions for men who are 12, 24, and 36 months beyond negative biopsy are reasonably accurate.

FIG. 3. Block diagram of a computer system useful to predict a positive repeat biopsy after a negative prostate biopsy and/or the timing for performing a repeat biopsy.

FIG. 4. Information processing system useful to predict a positive repeat biopsy after a negative prostate biopsy and/or the timing for performing a repeat biopsy.

FIG. 5. Machine readable medium useful to predict a positive repeat biopsy after a negative prostate biopsy and/or the timing for performing a repeat biopsy.

FIG. 6. Repeat biopsy nomogram used for validation study. Each scale position has corresponding prognostic points (top axis). The point values for all the predictor variables are determined consecutively and are summed to arrive at a total points value. This value is plotted on the total points axis and directly below it is the prediction for finding cancer. Age: use the age at time of present biopsy; DRE: current digital rectal examination result (N=negative, ≦½ lobe=suspicious nodule less than half a lobe, >½ lobe=suspicious nodule greater than half a lobe); negative cores previously taken: the cumulative cores previously removed; history of HGPIN and ASAP: affirmative when they have been found before in any biopsy session; PSA: the current serum PSA level; PSA slope: subtract the previous serum PSA measurement from the current PSA and divide this difference by the years between the PSA measurements; family history: affirmative when a first or second degree relative has been diagnosed with prostate cancer; months from initial negative biopsy: months from first biopsy to today, months from previous negative biopsy: month from last negative biopsy to the end of the study.

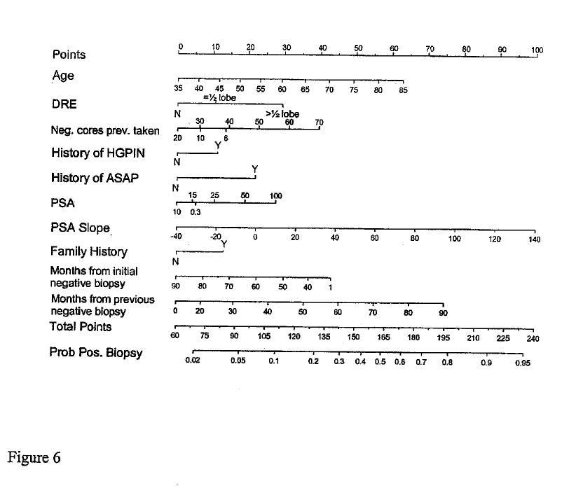

FIG. 7. Calibration of nomogram on validation data. The dotted line indicates the ideal reference line, where the predicted probability would perfectly match observed proportions. Triangles represent performance of the nomogram. Horizontal lines at the bottom of the plot denote the distribution of predicted probabilities.

### DETAILED DESCRIPTION OF THE INVENTION

The invention includes a method to predict prostate disease status after an initial negative biopsy and/or the timing for a repeat prostate biopsy in a patient with a previous negative prostate biopsy. In one embodiment, the method is particularly useful for evaluating patients at risk for prostate cancer following a negative prostate biopsy. For instance, factors including but not limited to age, DRE results, PSA, e.g., PSA density, percent free PSA, transition zone PSA, proPSA or BPSA, PSA slope, number of negative cores, family history of prostate cancer, history of HGPIN, history of ASAP, number of months since initial negative biopsy, number of months from previous negative biopsy, as well as markers other than PSA in physiological fluid, for instance, TGF-β₁, IL-6, IL6sR, IGF BP-2, IGF BP-3, UPA, UPAR, VEGF, sVCAM, proPSA or BPSA, parameters from a 12-core systemic biopsy of the prostate, or imaging, may be useful in predicting, for example, disease status or the timing for a repeat biopsy. Thus, such methods are useful in prognosis of prostate cancer. Moreover, such methods provide valuable means of monitoring the status of the patient. In addition to improving prognostication, knowledge of the disease status allows the attending physician to select the most appropriate follow up for the individual patient. It would be desirable to distinguish patients with a higher likelihood of developing prostate cancer or who should be monitored more closely. For example, patients with a high likelihood of a positive repeat biopsy can be monitored more frequently.

The present invention thus provides methods, apparatus and nomograms to predict a positive repeat prostate biopsy after a negative biopsy, i.e., the presence of prostate cancer, or the timing of a repeat biopsy using factors available prior to, from, and/or after an initial negative biopsy. In one embodiment, a nomogram predicts the probability of a positive repeat prostate biopsy after a negative biopsy. These nomograms can be used in clinical decision making by the clinician and patient, for example, to identify patients who may benefit from a repeat biopsy, and when that biopsy should be performed.

Accordingly, one embodiment of the invention is directed to a method for predicting a positive repeat prostate biopsy after a negative biopsy, and/or the timing of a repeat biopsy in a male at risk of or suspected of having prostate cancer. The method comprises correlating at least one and optionally a selected set of factors determined for each of a plurality of persons previously subjected to prostatic biopsies, at least the first of which was negative and one of which was positive, with the incidence of prostatic cancer for each person of the plurality of persons, so as to generate a functional representation of the correlation. The selected set of factors includes, but is not limited to, age, DRE, PSA, PSA slope, number of negative cores, family history, history of HGPIN, history of ASAP, months since initial negative biopsy, months from previous negative biopsy, PSA density, percent free PSA, transition zone PSA, markers in physiological fluid such as TGF-β₁, IL6sR, sVCAM, VEGF, UPAR, UPA, BPSA or proPSA, parameters form a 12-core systemic biopsy of the prostate, or imaging. An identical factor or set of factors determined from a patient having a negative prostate biopsy is matched to the functional representation so as to predict the probability of a positive repeat prostate biopsy or timing of repeat biopsy. The terms “correlation,” “correlate” and “correlating” include a statistical association between factors and outcome, and may or may not be equivalent to a calculation of a statistical correlation coefficient.

In one embodiment, the correlating includes accessing a memory storing the selected set of factors. In another embodiment, the correlating includes generating the functional representation and displaying the functional representation on a display. In one embodiment, the displaying includes transmitting the functional representation from a source. In one embodiment, the correlating is executed by a processor or a virtual computer program. In another embodiment, the correlating includes determining the factor or selected set of factors. In one embodiment, determining includes accessing a memory storing the factor or set of factors from the patient. In another embodiment, the method further includes transmitting the quantitative probability of a positive repeat prostate biopsy after a negative biopsy and/or the timing of a repeat biopsy. In yet another embodiment, the method further includes displaying the functional representation on a display. In yet another embodiment, the method further includes inputting the identical factor or set of factors for the patient within an input device. In another embodiment, the method further includes storing any of the factor or set of factors to a memory or to a database. In one embodiment, the functional representation is a nomogram and the patient is a male at risk of or suspected of having prostate cancer. In one embodiment, the plurality of persons includes persons having had at least two prostate biopsies, the first of which was negative and one of which was positive.

In one embodiment, the nomogram is generated with a Cox proportional hazards regression model (Cox, 1972, the disclosure of which is specifically incorporated by reference herein). This method can predict survival-type outcomes using multiple predictor variables. The Cox proportional hazards regression method estimates the probability of reaching a certain end point, such as disease recurrence, over time. In another embodiment, the nomogram may be generated with a neural network model (Rumelhart et al., 1986, the disclosure of which is specifically incorporated by reference herein). This is a non-linear, feed-forward system of layered neurons which backpropagate prediction errors. For instance, an artificial neural network (Dreiseitl et al., 2002, the disclosure of which is specifically incorporated by reference herein) or a Bayesian neural network (Barlow et al., 2001; Hauben et al., 2003, the disclosures of which are specifically incorporated by reference herein) may be employed. In another embodiment, the nomogram may be generated with a recursive partitioning model (Breiman et al., 1984, the disclosure of which is specifically incorporated by reference herein). In yet another embodiment, the nomogram is generated with support vector machine technology (Cristianni et al., 2000; Hastie, 2001, the disclosures of which are specifically incorporated by reference herein). In yet another embodiment, classification and regression trees (CART) can be used (Province et al., 2001; Begg, 1986, the disclosures of which are specifically incorporated by reference herein). Other models known to those skilled in the art may alternatively be used. In one embodiment, the invention includes the use of software that implements Cox regression models or support vector machines to predict a positive repeat prostate biopsy after a negative biopsy having prostate cancer, and/or the timing of a repeat biopsy.

A nomogram of the invention may include an apparatus for predicting a probability of a positive repeat prostate biopsy after a negative biopsy having prostate cancer and/or whether or not to repeat a biopsy. The apparatus includes a correlation of one or more factors determined for each of a plurality of persons previously subjected to two or more biopsies with the probability a positive repeat prostate biopsy after a negative biopsy for each person of the plurality of persons, the factors including but not limited to age, DRE, PSA, PSA slope, number of negative cores, family history, history of HGPIN, history of ASAP, months since initial negative biopsy, months from previous negative biopsy, levels of one or more of TGF-β₁, IL6sR, sVCAM, VEGF, PSA, UPAR, UPA, proPSA and/or BPSA, parameters from at least a 6 core biopsy, e.g., one with 7, 8, 9, 10, 11, 12 or more cores; and a means for matching an identical factor or set of factors determined from the patient to the correlation to predict the probability a positive repeat substrate biopsy after a negative biopsy having prostate cancer and/or the timing of a repeat biopsy.

The nomogram or functional representation may assume any form, such as a computer program, e.g., in a hand-held device, world-wide-web page, e.g., written in FLASH, or a card, such as a laminated card. Any other suitable representation, picture, depiction or exemplification may be used. The nomogram may comprise a graphic representation and/or may be stored in a database or memory, e.g., a random access memory, read-only memory, disk, virtual memory or processor.

The apparatus comprising a nomogram may further comprise a storage mechanism, wherein the storage mechanism stores the nomogram, an input device that inputs the identical set of factors determined from a patient into the apparatus, and a display mechanism, wherein the display mechanism displays the quantitative probability of a positive repeat prostate biopsy after a negative biopsy having prostate cancer and/or the timing of a repeat biopsy. The storage mechanism may be random access memory, read-only memory, a disk, virtual memory, a database, and a processor. The input device may be a keypad, a keyboard, stored data, a touch screen, a voice activated system, a downloadable program, downloadable data, a digital interface, a hand-held device, or an infra-red signal device. The display mechanism may be a computer monitor, a cathode ray tub (CRT), a digital screen, a light-emitting diode (LED), a liquid crystal display (LCD), an X-ray, a compressed digitized image, a video image, or a hand-held device. The apparatus may further comprise a display that displays the quantitative probability of a positive repeat prostate biopsy after a negative biopsy having prostate cancer and/or the timing of a repeat biopsy, e.g., the display is separated from the processor such that the display receives the quantitative probability of a positive repeat prostate biopsy after a negative biopsy having prostate cancer and/or the timing of a repeat biopsy. The apparatus may further comprise a database, wherein the database stores the correlation of factors and is accessible by the processor. The apparatus may further comprise an input device that inputs the identical set of factors determined from the patient into the apparatus. The input device stores the identical set of factors in a storage mechanism that is accessible by the processor. The apparatus may further comprise a transmission medium for transmitting the selected set of factors. The transmission medium is coupled to the processor and the correlation of factors. The apparatus may further comprise a transmission medium for transmitting the identical set of factors determined from the patient, preferably the transmission medium is coupled to the processor and the correlation of factors. The processor may be a multi-purpose or a dedicated processor. The processor includes an object oriented program having libraries, said libraries storing said correlation of factors.

In one embodiment, the nomogram comprises a graphic representation of a probability that a male at risk of or suspected of having prostate cancer a positive repeat prostate biopsy after a negative biopsy, and/or the timing of a repeat biopsy comprising a substrate or solid support, and a set of indicia on the substrate or solid support, the indicia including one or more of a PSA slope line, months from previous negative biopsy line, an age line, months from initial negative biopsy line, number of previous negative cores line, a PSA level line, a DRE line, a history of ASAP line, a history of HGPIN line, and/or family history line, a points line, a total points line and a predictor line, wherein each line has values on a scale which can be correlated with values on a scale on the points line. The total points line has values on a scale which may be correlated with values on a scale on the predictor line, such that the value of each of the points correlating with the indicia can be added together to yield a total points value, and the total points value correlated with the predictor line to predict the a positive repeat prostate biopsy after a negative biopsy having prostate cancer and/or the timing of a repeat biopsy. The solid support is preferably a laminated card that can be easily carried on a person.

In addition to assisting the patient and physician in selecting an appropriate course of monitoring, the nomograms of the present invention are also useful in clinical trials to identify patients appropriate for a trial, to quantify the expected benefit relative to baseline risk, to verify the effectiveness of randomization, to reduce the sample size requirements, and to facilitate comparisons across studies.

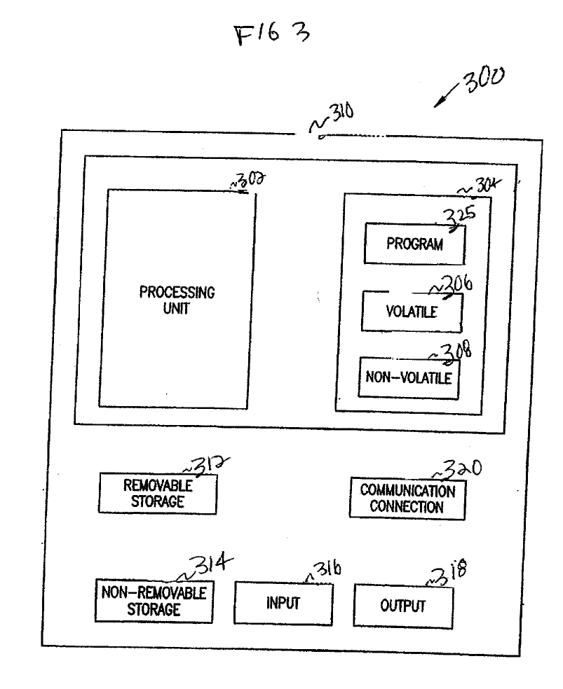

A block diagram of a computer system that executes programming for predicting a prognosis probability is shown in FIG. 3. A general computing device in the form of a computer 310, may include a processing unit 302, memory 304, removable storage 312, and non-removable storage 314. Memory 304 may include volatile memory 306 and non-volatile memory 308. Computer 310 may include—or have access to a computing environment that comprises—a variety of computer-readable media, such as volatile memory 306 and non-volatile memory 308, removable storage 312 and non-removable storage 314. Computer storage comprises RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or any other medium capable of storing computer-readable instructions. Computer 310 may include or have access to a computing environment that comprises input 316, output 318, and a communication connection 320. Input 316 may include one or several devices such as a keyboard, mouse, touch screen, and stylus. Output 318 may include one or several devices such as a video display, a printer, an audio output device, a touch stimulation output device, or a screen reading output device. The computer may operate in a networked environment using a communication connection 320 to connect to one or more remote computers. The remote computer may include a personal computer, server, router, network PC, a peer device or other common network node, or the like. The communication connection 320 may include a Local Area Network (LAN), a Wide Area Network (WAN) or other networks. The communication connection 1020 may be over a wired network, wireless radio frequency network, or an infrared network. Further, in some embodiments, the network may be a combination of several connection technologies including wired, RF, and/or infrared.

Computer-readable instructions stored on a computer-readable medium are executable by the processing unit 302 of the computer 310. A hard drive, CD-ROM, and RAM are some examples of articles including a computer-readable medium. The computer-readable instructions allow computer system 1000 to provide generic access controls in a computer network system having multiple users and servers, wherein communication between the computers includes utilizing TCP/IP, COM, DCOM, XML, Simple Object Access Protocol (SOAP), and Web Services Description Language (WSDL), and other related connection communication protocols and technologies that will be readily apparent to one of skill in the relevant art.

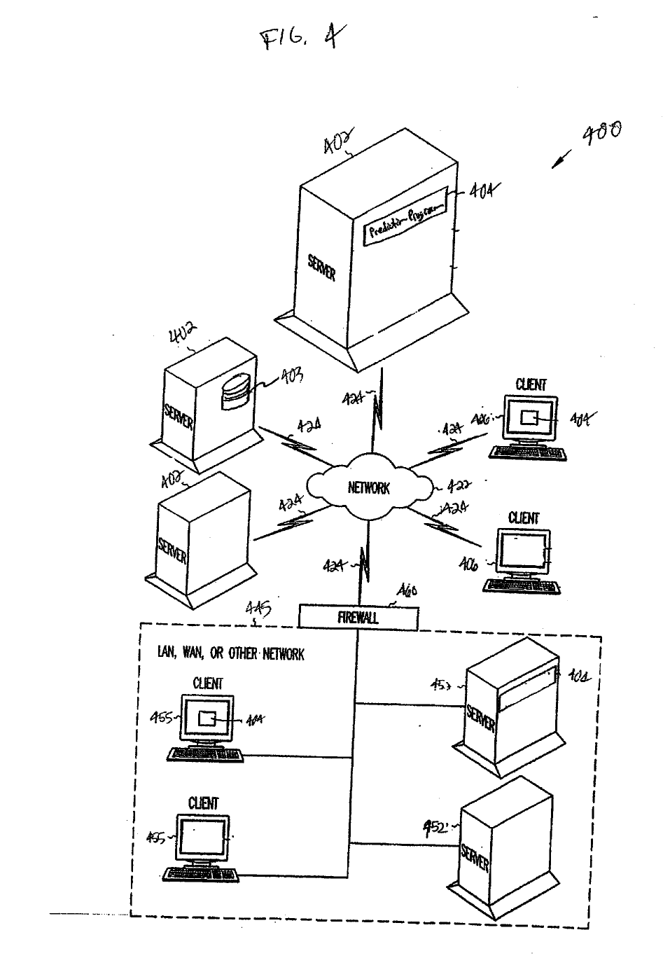

FIG. 4 shows an exemplary embodiment of an information processing system 400 that provides for transfer of data between multiple devices. This embodiment of system 400 comprises multiple servers 402, client work stations 406, the servers 402 and client workstations 406 operatively connected via communication lines 424 to a network 422. In one embodiment, network 422 includes the Internet, or other type of public or private network that allows data transfer. Communication lines 424 may be any type of communication medium, such as telephone lines, cable, optical fiber, wireless, or any other communication medium that allows data transfer between devices coupled to the network.

In some embodiments, one or more of the servers 402 hold a prediction program 404, which is available for download to the other servers 402 and workstation clients 406 connected 424 to the network 422.

In some other embodiments, prediction program 1104 is executable on a server 402 wherein the prediction program executes in response to stimulation received from a client 404 using a Hyper-Text Transfer Protocol (HTTP or HTTPS). In one such embodiment, prediction program 404 accepts input from a client, executes, and outputs a prognosis prediction in a markup language such as Hyper-Text Markup Language (HTML) or extensible Markup Language (XML).

In some embodiments, system 400 may be implemented with servers 402 utilizing one of many available operating systems. Servers 402 may also include, for example, machine variants such as personal computers, handheld personal digital assistants, RISC processor computers, MIP single and multiprocessor class computers, and other personal, workgroup, and enterprise class servers. Further, servers 402 may also be implemented with relational database management systems 403 and application servers. Other servers 402 may be file servers.

Client workstations 402 within embodiments of system 400, may include personal computers, computer terminals, handheld devices, and multifunction mobile phones. Client workstations 402 include software thereon for performing operations in accordance with stimulation received from a user and signals received from other computing devices on the network 422. Further, a client workstation 402 may include a web browser for displaying web pages.

The network 422 within some embodiments of a system 400 may include a Local Area Network (LAN), Wide Area Network (WAN), or other similar network 445 connected 424 network 422. Network 422 may itself be a LAN, WAN, the Internet, or other large scale regional, national, or global network or a combination of several types of networks. Some embodiments of system 400 include a LAN, WAN, or other similar network 445 that utilizes one or more servers 452 and clients 455 behind a firewall 460 within the LAN, WAN, or other similar network 445.

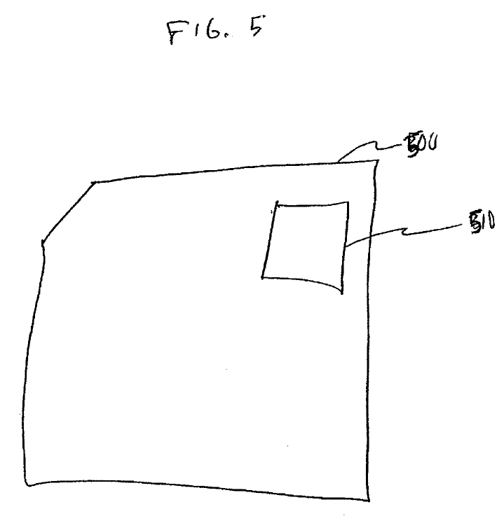

FIG. 5 shows an exemplary embodiment of a machine-readable medium 500 with operable instructions 510 thereon for performing the methods described herein on an appropriately configured information processing device. Such devices include in various embodiments personal computers including desktop, laptop, and tablet computers. Some further embodiments include handheld devices utilizing Palm/OS or Windows CE.

The invention will be further described by the following non-limiting examples.

### EXAMPLE I

To develop a prognostic model or nomogram useful for counseling patients in the decision to repeat prostate biopsy, previous clinical and histological patient findings were analyzed for biopsied patients (Table 1) and selected patients with one or more prior initially negative biopsy sessions.

Materials and Methods

Patients. 687 patients who underwent a total of 1,348 consecutive prostate biopsy sessions were studied. Of these patients, 344 had cancer on the initial biopsy and were excluded from the study. The remaining 343, who had at least one initial negative biopsy, were selected for analysis. The indications to repeat biopsy and the number of cores obtained per biopsy session varied among physicians. The typical biopsy method employed in the present study was sextant biopsy, possibly with 2 transition zone biopsies, and directed biopsy when a suspicious area was seen on transrectal ultrasound. All repeat biopsies included 2 transition zone biopsies. In the last approximate quarter of data collection, the extended biopsy protocol was performed by several of the attending physicians, including 12 cores from the peripheral core and 2 transitional zone biopsies. The number of cores obtained per repeat biopsy session was 6 to 22. Prostate biopsies were done under transrectal ultrasound guidance with a biplanar 6.5 MHz probe using a biopsy gun and an 18 gauge Tru-Cut (Travenol Laboratories, Deerfield, Ill.) biopsy needle with the patient under local anesthesia.

Variables. Each core from each biopsy session was categorized as normal, with HGPIN and/or ASAP, or with prostate cancer according to predefined criteria (Epstein et al., 2001; Epstein, 1998). The number of cores per prostate biopsy set was analyzed as a separate variable. There was no minimum number of cores required for inclusion of a particular prostate biopsy session in the study.

The clinical variables collected prior to each prostate biopsy session were patient age, presence or absence of family history of prostate cancer, serum PSA (Hybritech, Inc., San Diego, Calif.), PSA slope in ng/ml yearly (the difference between the most current serum PSA measured minus serum PSA at the previous biopsy divided by elapsed time in years), PSA density, DRE findings (negative, suspicious for a nodule less than or larger than half a lobe), months elapsed between the first negative biopsy and each subsequent biopsy session, cumulative number of negative cores previously obtained, and history of HGPIN and ASAP. In this way, several clinical and pathological parameters were analyzed together to determine the collective predictive ability regarding the presence of cancer.

Statistical analysis. Descriptive statistics for all variables were calculated. Tests of significance for the association of each variable with a positive biopsy were performed using a time dependent covariate Cox regression model. The nomogram was constructed using the Design library of S-plus (Insightful Corp., Seattle, Wash.) software. Discrimination was calculated as the concordance index. Calibration was assessed graphically. Discrimination and calibration were performed using jackknifing procedures to obtain less biased estimates.

Results

A total of 1004 biopsy sessions were performed in 343 patients, of which 661 were repeat sessions. Table 1 lists the characteristics of the group. A mean of 2.9 biopsy sessions per patient were performed.

TABLE 1
Clinical characteristics of 343 patients
with repeat prostate biopsy sessions

Variable

| Mean age ± SD (range) | 62.1 ± 7.6 | | (38-81) |
|---|---|---|---|
| No. family history (%):* | | | |
| Neg | 283 | (83) | |
| Pos | 60 | (7) | |
| No. DRE (%):* | | | |
| Neg | 275 | (80%) | |
| Pos ½ lobe or less | 61 | (18%) | |
| Pos greater than ½ lobe | 7 | (2%) | |
| Mean No. HGPIN history (%):† | | | |
| Neg | 254 | (74) | |
| Pos | 89 | (26) | |
| Mean No. ASAP history (%):† | | | |
| Neg | 298 | (97) | |
| Pos | 45 | (13) | |
| Mean No. normal histology (%):† | 227 | (66.2) | |

| | | |
|---|---|---|
| Mean mos ± SD from first to last | 24.65 ± 20 | (0.17-97) |
| biopsy session (range) | | |
| Mean no. biopsy sessions ± SD | 2.92 ± 1.36 | (2-12) |
| (range)† | | |
| Mean No. cores/biopsy ± SD | 9.15 ± 2.78 | (6-22) |
| (range)† | | |
| Mean cumulative No. cores ± SD | 25.2 ± 11.43 | (12-87) |
| (range)† | | |
| Mean ng/ml PSA ± SD (range)† | 8.4 ± 9.6 | (0.28-123) |
| Mean ng/ml/yrs PSA slope ± SD | 1.30 ± 18.3 | (−38-124.3) |
| (range) | | |

*Findings of initial biopsy session only.
†Values of 661 repeat biopsy sessions.

Each patient underwent at least 2 biopsy sessions, 47% had 3 and 45% had 4 or more biopsy sessions. Table 2 shows the positive prostate biopsy rate according to the number of biopsy sessions and several other variables.

TABLE 2
Distribution of cases with prostate cancer according to the
number of biopsy sessions performed and other variables

| | Total No./No. Ca (%) | | | | | |
|---|---|---|---|---|---|---|
| Variable | Biopsy 2 | | Biopsy 3 | | Biopsy 4 | |
| DRE: | | | | | | |
| Neg | 274/48 | (17.5) | 133/16 | (12) | 66/9 | (14) |
| Pos ½ | 62/16 | (25.8) | 26/4 | (15) | 15/3 | (20) |
| lobe or less | | | | | | |
| Pos greater than | 7/3 | (43) | 3/2 | (67) | 1/0 | |
| ½ lobe | | | | | | |
| PSA (ng/ml): | | | | | | |
| Less than 2.5 | 20/2 | (10) | 7/2 | (28) | 4/2 | (50) |
| 2.5-4 | 24/7 | (29) | 12/2 | (16) | 2/0 | |
| 4-10 | 230/41 | (18) | 98/11 | (11) | 40/4 | (10) |
| Greater than 10 | 69/17 | (25) | 45/7 | (15.5) | 35/5 | (14) |
| PSA slope | | | | | | |
| (ng/ml/yr): | | | | | | |
| Less than 0.75 | 207/39 | (19) | 101/12 | (12) | 40/7 | (17) |
| 0.75 or Greater | 136/28 | (21) | 61/10 | (16) | 42/5 | (12) |
| Previous HGPIN: | | | | | | |
| Neg | 254/47 | (18.5) | 95/11 | (11.5) | 40/3 | (7.5) |
| Pos | 89/20 | (22) | 67/11 | (16) | 42/8 | (19) |
| Previous ASAP: | | | | | | |
| Neg | 298/53 | (18) | 137/17 | (12) | 67/8 | (12) |
| Pos | 45/14 | (31) | 25/5 | (20) | 15/3 | (20) |
| Normal histology | 227/40 | (18) | 112/16 | (14) | 53/4 | (7) |
| Totals | 343/67 | (19.5) | 162/22 | (13.5) | 82/12 | (15) |

Results of cases with 5 or more biopsy sessions are not shown because of low numbers.

ASAP and HGPIN were found in 116 patients and these findings were concordant in 18.

Table 3 shows the results of the Cox regression model. Of all variables, the cumulative number of negative cores previously obtained, history of HGPIN, history of ASAP, and PSA slope were associated with repeat biopsy findings on multivariable analysis (all p<0.05).

TABLE 3
Results of the time dependent covariable Cox regression
model of predictors for positive repeat prostate biopsy

| Nomogram Predictors | p Value |
|---|---|
| Age | 0.369 |
| Prostate Ca family history | 0.065 |
| DRE | 0.073 |
| Cumulative No. previous neg cores | 0.002 |
| PSA | 0.510 |
| PSA slope | 0.001 |
| Previous HGPIN | 0.001 |
| Previous ASAP | 0.001 |

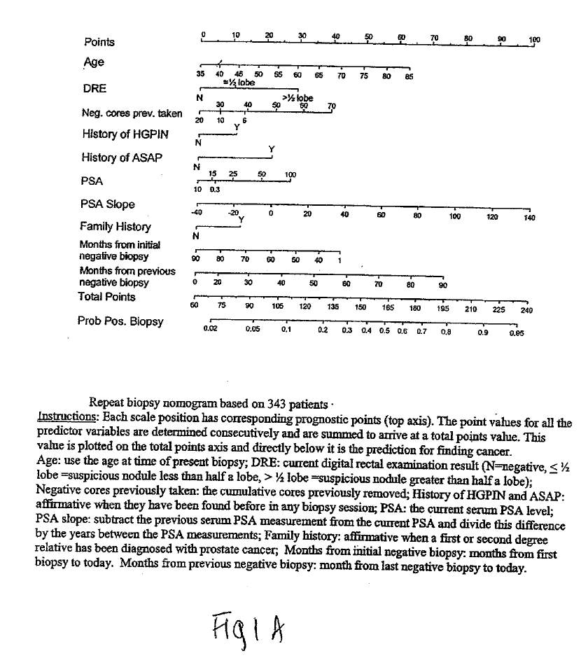

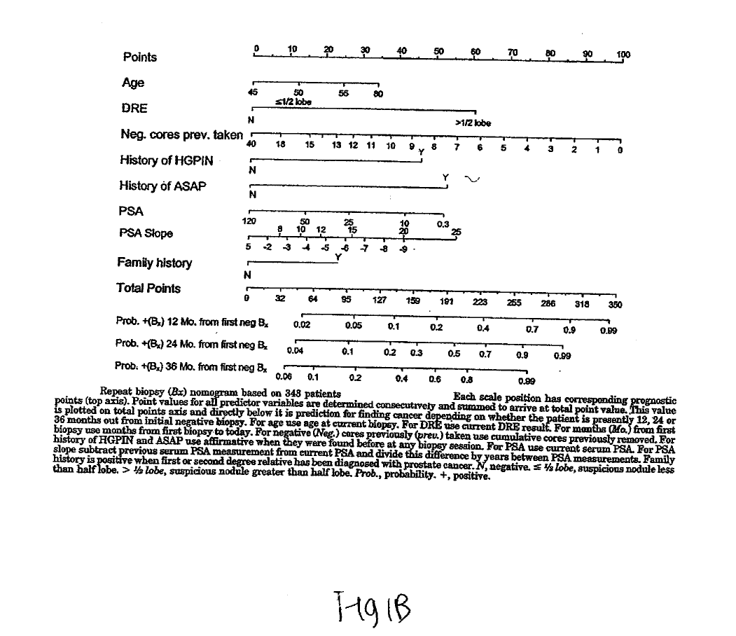

A nomogram was constructed incorporating all predictors (FIG. 1). Although some variables were not statistically significant predictors of a positive repeat biopsy, they were retained in the nomogram because omitting them from the model tended to decrease accuracy. However, PSA density was missing from many biopsy sessions and omitting it did not harm predictive accuracy. Thus, for practical reasons it was omitted from the nomogram.

The nomogram is used by first locating the patient position on each predictor variable scale. Each scale position has corresponding prognostic points (top axis). Point values for all the predictor variables are determined consecutively and summed to arrive at a total point value. This value is located on the total point axis and directly below is the prediction of finding cancer. If the patient is 12 months from the initial negative biopsy, one would draw a line down the 12-month axis to determine the probability of a positive biopsy today.

The nomogram was evaluated for its ability to discriminate among patient risks of being found with cancer on repeat biopsy. It was measured as the concordance index, which was 0.70 using the jackknife predictions. This jackknife derived concordance index addresses only its ability to discriminate among patients.

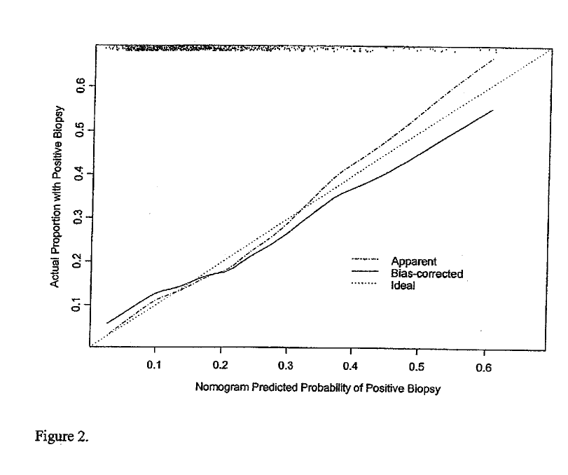

It does not address the calibration accuracy of the monogram (i.e., the absolute error of its prediction). FIG. 2 shows this calibration by plotting the nomogram predicted probability for positive repeat biopsy against the patient observed or actual probability for each of the 3 time points of prediction. This calibration error was determined by jackknifing. In general, the performance of the nomogram appears to be reasonably accurate. Its least accurate prediction appears to be for a man with a high predicted probability 36 months after the initial negative biopsy. This prediction is too high and should be decreased in practice.

Finally, the nomogram predictive accuracy was compared with that achieved by using recommendations based on individual risk factors from the literature (Table 4). Each of these variables individually predicted prostate cancer on repeat biopsies with a concordance index no greater than 0.63, which was lower than the 0.70 achieved using the nomogram.

TABLE 4
Comparison of nomogram results and other predictors

| Predictors | Accuracy (concordance index) |
|---|---|
| HGPIN history⁷ | 0.63 |
| ASAP history⁷ | 0.61 |
| PSA less than 4, 4-10, 10-20, | 0.53 |
| greater than 20 ng/ml⁹ | |
| PSA density⁴ greater than 0.15 | 0.52 |
| PSA slope4 greater than 0.75 ng/ml/yr | 0.53 |
| The present nomogram | 0.70 |

Discussion

There are different recommendations for determining the patient risk for prostate cancer on an initial biopsy based on several predictors, such as serum PSA, age, DRE findings and percent free PSA. Recently, an artificial neural network was constructed for predicting biopsy results (Carlson et al., 1998; Djavan et al., 2002). A nomogram has been constructed for patients with a serum PSA of below 4 ng/ml (Eastham et al., 1999) and a study has been performed of PSA derivatives (Djavan et al., 1999). The 2 studies estimate the risk of finding cancer in this select group of patients.

Determining the patient risk for prostate cancer on repeat biopsy has received less attention. The detection rate of subsequent biopsies is lower. Roehl et al. (2002) reported that the rate decreased from 30% in the first biopsy to 7% after 6 biopsy sessions. In the present study, the rate decreased from 19.5% in the second biopsy to 13.5% after 5 or more biopsy sessions. Apparently if one continues to do serial prostate biopsies on each patient, the overall rate of prostate cancer detection remains at approximately 7% (O'Dowd et al., 2000; Roehl et al., 2002).

Determining who needs another biopsy is difficult. Others have analyzed markers or risk factors that confer a significant risk for cancer discovered on a repeat biopsy. Most studies relied on 1 or 2 markers to determine risk. Dajavan et al. (2000) found that percent free PSA and transition zone PSA density predicted better than total serum PSA and PSA density in patients with a total serum PSA of 4 to 10 ng/ml with an area under the curve (AUC) of 74% and 69%, respectively. Similarly, Fowler et al. (2000) found that percent free PSA was the best predictor of cancer compared with the other PSA derivatives. In contrast, Keetch et al. (1994) noted that PSA density and PSA slope were the best predictors of cancer on repeat biopsy. Some studies indicated that baseline risk in patients at low risk is sufficiently high that one cannot define a subset for whom repeat biopsy is unnecessary (Fleshner et al., 1997). Keetch et al. (1993) mentioned that patients with persistently elevated serum PSA after an initial negative biopsy should undergo at least 1 repeat biopsy. Although the present nomogram covers most of the well-known risk factors that can predict the presence of cancer, there are other variables that were not analyzed but have previously proven useful, including percent free PSA and transition zone PSA density (O'Dowd et al., 2000; Fowler et al., 2000; Djavan et al., 2000). However, Hayek et al. (1994) did not note a statistical difference using PSA density and percent free PSA to identify patients with and without cancer in a series of 61 with repeat biopsy. Percent free PSA and ultrasound volume were not available for many of the patients in the present study and while the literature is conflicting, incorporating these additional predictors may result in a nomogram with increased accuracy.

Cancer rates in men with ASAP and HGPIN are 21% to 57.1% and 27% to 79%, respectively, reflecting that other factors also have a role in determining the likelihood of cancer (Epstein et al., 2001). Recently O'Dowd et al. (2000) and Vis et al. (2001) questioned the relationship between a previous diagnosis of HGPIN and cancer on subsequent biopsy by showing that similar cancer rates were found in patients with previous benign prostatic hyperplasia only. O'Dowd et al. (2000) discovered prostate cancer in 40% of their patients with ASAP in the initial biopsy compared with 22% who had HGPIN and 19.8% in whom normal tissue was seen initially. By logistic regression analysis using age, previous histological findings, free PSA and total PSA, their model predicted cancer in a second biopsy with an uncorrected AUC of 0.69. Their model is strictly for the second biopsy and its accuracy is unclear after correcting for over fit.

By simultaneously incorporating multiple variables, the present nomogram had dramatic effects on the predicted probability of having prostate cancer. For example, a patient with a positive history of ASAP alone has been reported to have a 50% chance of having undiscovered cancer (Epstein et al., 2001). However, according to the present nomogram, if that patient has low serum PSA, has a minimal change in serum PSA, is relatively young and has had more than 10 cores obtained previously, his nomogram estimated probability of having undiscovered cancer would be 12% at 12 months after initial biopsy and 23% at 24 months. In comparison to the predictive accuracy of several individual variables according to previously reported analysis, the present nomogram performed better (AUC 0.63 or less vs. 0.70, Table 4). The nomogram obviously uses considerably more information than any single risk factor alone.

Another use for the nomogram is to define the timing of repeat biopsy. For example, if a 65-year-old patient has a small but suspicious nodule on DRE, has had 3 sextant biopsy sessions previously, has a positive history of ASAP, a current PSA of 20 ng/ml, a PSA slope of 10, and a close relative with prostate cancer, he has a 40% and 59% probability of having cancer in a fourth biopsy if it is 24 or 36 months after the initial biopsy session, respectively. Under those circumstances, a physician likely would consider repeating the biopsy again. However, if this patient does not have ASAP or a family history of prostate cancer, has negative DRE findings and the PSA slope is 1 even with the same elevated total PSA, the probability of cancer if it is currently 24 or 36 months after initial biopsy is 5% and 11%, respectively. The best option for this patient is to follow him with periodic serum PSA measurements and DRE, and repeat the biopsy if a new clinical indication develops. This nomogram is also useful for visualizing the association that that each variable has with the prediction of cancer on repeat biopsy. Increasing serum PSA slope or negative values, increasing patient age, a progressively larger suspicious nodule on DRE and a decreasing number of cores obtained previously substantially increase the predicted probability of discovering cancer (FIG. 1).

There have been studies relying on imaging to identify patients with prostate cancer after an initial negative prostate biopsy. Perrotti et al. (1999) classified their patients with low, intermediate or high suspicion on endorectal magnetic resonance imaging and found cancer on repeat biopsies in 5.6%, 12.5% and 71.4%, respectively. This observation suggests that imaging may have an important role in identifying patients suspected with cancer after a previous negative biopsy by helping avoid another biopsy if suspicion is low (94.4% negative predictive value in the low risk group). Magnetic resonance imaging could also offer the possibility of directing the biopsy to suspicious lesions.

Another important issue that the present nomogram does not address is the rate of indolent cancer found on repeat biopsies. Indolent cancer is defined as a tumor of less than 0.5 cm³ that is organ confined and without Gleason pattern 4 or 5. There is evidence that prostate cancer found after previous negative biopsies has a higher probability of being indolent (Epstein et al., 1999). This issue must be considered before deciding to perform a subsequent biopsy and a predictive model for indolent cancer would be invaluable for this purpose.

Finally, the decision to repeat prostate biopsy must be made on a case by case basis. It should be done according to the predicted probability for prostate cancer, patient clinical history, the minimal risk that the patient is willing to accept before deciding to undergo another biopsy session and the likelihood of detecting indolent disease.

In conclusion, a nomogram was developed that predicts the probability of finding prostate cancer in patients who have undergone one or more prior negative biopsies. This nomogram is based on clinical and histological features of previous negative biopsies. The present nomogram provides accurate probability and it may also improve clinical judgment when deciding if a patient requires another biopsy.

### EXAMPLE II

Methods and Materials

Patients. Validation data representing men undergoing one or more repeat transrectal ultrasound guided prostate biopsies after an initial negative biopsy were obtained from the Brooklyn Veterans Administration Medical Center. There were 416 patients who totaled 1026 biopsies. Patients with missing PSA values (n=5), missing DRE results (n=58), or missing family history (n=152) were excluded from the analysis, leaving 230 patients with complete records in the validation data set. Of these patients, 126 were black (54.8%), 88 were white (38.2%), and 16 were of other races (7.0%). Standard sextant biopsies were performed up to 1998 while 12 core biopsies which incorporated the lateral and medial aspects of the base, mid, and apex of the peripheral zones were performed from 1999 to 2003. All patients with an initial negative biopsy did not undergo repeat biopsy. Indications for repeat biopsy at this institution included presence of HGPIN and/or ASAP on a previous biopsy, persistently elevated PSA or positive DRE, and PSA slope greater than 0.75 ng/ml/year.

Variables required for the nomogram. The clinical variables obtained prior to each patient's biopsy session were patient age, serum PSA, PSA slope in ng/ml/year (the difference between the most recent PSA minus PSA at the previous biopsy divided by elapsed time in years), DRE findings (negative, presence of a nodule less than or larger than half a lobe), months from initial negative biopsy to each subsequent biopsy, and cumulative number of previous negative cores. Results recorded from each biopsy session included presence of normal histology, presence of cancer, or presence of HGPIN and/or ASAP as defined (Epstein et al., 2001; Epstein, 1998). The number of cores taken at each biopsy session as well as the cumulative number of cores taken per patient was also recorded.

Statistical analysis. The predicted probability of positive repeat biopsy was calculated for each patient using the logistic regression model underlying the nomogram. The concordance index, identical to the nonparametric area under the receiver operating characteristic curve, was computed for these predictions. Biopsy sessions were sorted and grouped by their predicted probabilities, and the proportion of positive men in each group was plotted against the mean predicted probability for each group.

Results

A total of 586 biopsies were performed in the 230 patients with 356 of these occurring as repeat sessions. The characteristics of the validation group and the original nomogram population were compared and are reported in Table 5. There was a mean of 2.51 biopsy sessions per patient. HGPIN was present in 32 patients, and ASAP was present in 71 patients with 13 patients having both concomitantly. Prostate cancer was eventually found in 78 patients. Table 7 shows the positive biopsy rate stratified by the number of biopsies as well as several of the variables studied.

TABLE 5
Clinical characteristics of the cohorts
with repeat prostate biopsy sessions

| Variable | BVA | | MSKCC | |
|---|---|---|---|---|
| Mean age ± SD | 66.3 ± 7.1 | (38-81) | 62.1 ± 7.6 | (38-81) |
| (range)† | | | | |
| No. family | | | | |
| history (%):* | | | | |
| Neg | 208 | (90) | 283 | (83) |
| Pos | 22 | (10) | 60 | (17) |
| No. DRE (%):* | | | | |
| Neg | 185 | (80.4) | 275 | (80) |
| Pos ½ | 40 | (17.4) | 61 | (18) |
| lobe or less | | | | |
| Pos greater than | 5 | (2.2) | 7 | (2) |
| ½ lobe | | | | |
| No. HGPIN | | | | |
| history (%):† | | | | |
| Neg | 198 | (86) | 254 | (74) |
| Pos | 32 | (14) | 89 | (26) |
| No. ASAP | | | | |
| history (%):† | | | | |
| Neg | 159 | (69) | 298 | (87) |
| Pos | 71 | (31) | 45 | (13) |
| No. normal | 139 | (60.4) | 227 | (66.2) |
| histology (%):† | | | | |
| Mean mos ± SD | 33.2 ± 22.8 | (1-97) | 24.65 ± 20 | (0.17-97) |
| from first to last | | | | |
| biopsy session | | | | |
| (range) | | | | |
| Mean No. biopsy | 2.56 ± 0.87 | (2-7) | 2.92 ± 1.36 | (2-12) |
| sessions ± SD | | | | |
| (range)† | | | | |
| Mean cumulative | 17.89 ± 7.22 | (12-54) | 25.2 ± 11.43 | (12-87) |
| No. cores ± SD | | | | |
| (range)† | | | | |
| Mean ng/ml | 10.3 ± 9.7 | (0.7-89.8) | 8.4 ± 9.6 | (0.28-123) |
| PSA ± SD | | | | |
| (range)† | | | | |
| Mean ng/ml/yr | 2.18 ± 1.22 | (−26-145) | 1.30 ± 18.3 | (−38-124.3) |
| PSA slope ± SD | | | | |
| (range)† | | | | |

Abbreviations: BVA, Brooklyn Veterans Administration Medical Center; MSKCC, Memorial Sloan-Kettering Cancer Center.
*Findings of initial biopsy session only.
†Findings among all biopsy sessions.

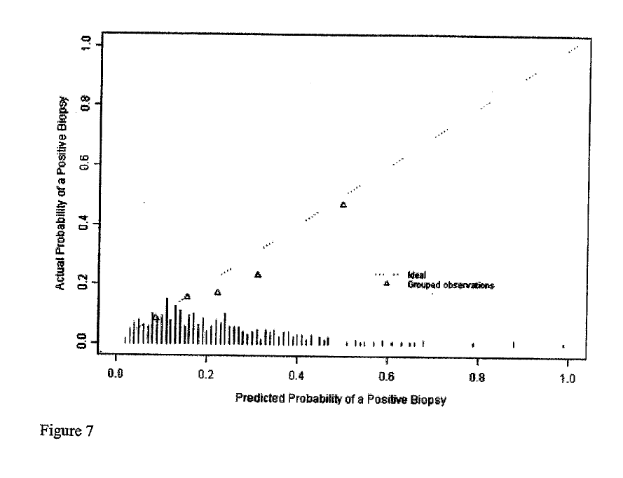

The area under the receiver operating characteristic curve (AUC) for the VA study group was 0.71. FIG. 7 shows the calibration of the nomogram as applied to the validation dataset. Biopsy sessions were arranged into equally sized groups with their mean predicted probabilities of a positive biopsy (x-axis) compared against the observed proportion in each group with a positive biopsy (y-axis). The nomogram appeared to perform reasonably well with the VA data set.

Finally, the predictive accuracy of the nomogram in the present study population was compared with that achieved by using recommendations based on individual risk factors reported in the literature (Table 6). Each of these risk factors predicted prostate cancer on repeat biopsy with the highest AUC being 0.64. This was lower than the 0.71 reached using the present nomogram.

TABLE 6
Distribution of cases with prostate cancer according to the
number of biopsy sessions performed and other variables

| | Total No./No. Ca (%) | | | | | |
|---|---|---|---|---|---|---|
| Variable | Biopsy 2 | | Biopsy 3 | | Biopsy 4 | |
| DRE: | | | | | | |
| Neg | 190/29 | (15) | 75/15 | (20) | 23/5 | (22) |
| Pos ½ lobe or less | 34/13 | (38) | 12/5 | (42) | 2/1 | (50) |
| Pos greater than ½ lobe | 6/6 | (100) | 1/1 | (100) | 1/1 | (100) |
| PSA (ng/ml): | | | | | | |
| Less than 4 | 10/1 | (10) | 0/0 | | 0/0 | |
| 4-10 | 146/19 | (13) | 47/9 | (19) | 7/0 | |
| Greater than 10 | 74/28 | (38) | 41/12 | (29) | 19/7 | (37) |
| PSA slope (ng/ml/yr): | | | | | | |
| Less than 0.75 | 107/8 | (7) | 34/7 | (21) | 8/1 | (12.5) |
| 0.75 or Greater | 123/40 | (33) | 54/14 | (26) | 18/6 | (33) |
| Previous HGPIN: | | | | | | |
| Neg | 210/43 | (20) | 73/18 | (25) | 18/6 | (33) |
| Pos | 20/5 | (25) | 15/3 | (20) | 8/1 | (12.5) |
| Previous ASAP: | | | | | | |
| Neg | 176/32 | (18) | 52/10 | (19) | 15/3 | (20) |
| Pos | 54/16 | (30) | 36/11 | (31) | 11/4 | (36) |
| Totals | 230/48 | (21) | 88/21 | (24) | 26/7 | (27) |

Results of cases with 5 or more biopsy sessions are not shown because of low numbers.

Discussion

In the repeat biopsy nomogram in Example I, the validation was performed only internally, without an external dataset. Validation using an external dataset is a more stringent test of a prediction model. For this purpose, the VA data set was used and performed with reasonable accuracy with an AUC of 0.71. This population represented a heterogeneous group of patients with a different racial composition from the first group of patients. Although the nomogram was not constructed to factor in potential racial differences in predicting biopsy positivity, it nonetheless appeared to perform well in the two patient sets.

Several studies have previously sought to define predictors for a positive prostate biopsy. Variables such as age, serum PSA, DRE results, percent free PSA, and PSA density have been used to construct strategies with the aim of defining patient risk for detecting prostate cancer (Djaven et al., 2002; Eastham et al., 1994). These studies have primarily focused on estimating risk in specific groups on initial biopsy.

Establishing an approach for the patient with an initial negative biopsy presents a challenge. Several authors have looked at combinations of variables that could predict risk of a positive repeat biopsy. The presence of HGPIN on initial biopsy leads to cancer in 23% to 100% of repeat biopsies while ASAP on initial biopsy leads to cancer in 29% to 60% of repeat biopsies (Keetch et al., 1996; Fowler et al., 2000; Djavan et al., 2000; Ellis et al., 1995; Park et al., 2001; Raviv et al., 1996; Keetch et al., 1995; O'Dowd et al., 2000; Ouyang et al., 2001; Cheville et al., 1997). In their analysis, Park et al. (2001) analyzed 45 patients with atypia and 43 with HGPIN on initial biopsy and found cancer in 51% of repeat biopsies in both groups. DRE and age were independent predictors of cancer within the atypia group while no variables were associated with cancer in the HGPIN group.

Fowler et al. (2000) examined results of repeat biopsies in a population with a similar racial composition as the present study. These patients were selected for initial biopsy based on abnormal DRE findings or a PSA of 4.0 ng/ml. On multivariate analysis, Fowler et al. found percent free PSA to be a strong predictor of repeat biopsy cancer detection (p=0.0003) with age also being significant (p=0.02) but not PSA density (p=0.65) or PSA velocity (p=0.94). Interestingly, they did not find HGPIN to be predictive of cancer in repeat biopsy which, as acknowledged by the authors, is contrary to many other published studies.

Djavan et al. (2000) also identified percent free PSA as a significantly better predictor of positive repeat biopsy than total serum PSA, PSA density, and transition zone PSA density. In their study, Djaven et al. found that PSA density and total serum PSA were not significant predictors of cancer on repeat biopsy. They focused on patients with a PSA from 4 to 10 ng/ml and recommended that men with a percent free PSA less than 30% or transition zone PSA density of 0.26 ng/ml/cc or greater undergo repeat biopsy. In opposition to some of the previous studies, Keetch et al. (1996) described PSA slope to be the most significant predictor of a positive repeat biopsy along with PSA density. They determined that men with a PSA density of 0.15 or greater and PSA slope of 0.75 ng/ml/year had 46% positive repeat biopsy rate compared to 13% in patients with PSA density less than 0.15 and PSA slope less than 0.75 ng/ml/year (p<0.0001).

When evaluating predictive accuracy of the previous individual variables against the present nomogram using the VA data set, the nomogram performed better (AUC 0.64 or less vs. 0.71, Table 7). As mentioned previously, the present nomogram incorporates some of the individual risk factors identified as predicting cancer on repeat biopsy. As a result, it is not surprising that it would predict cancer on repeat biopsy better than any individual risk factor alone.

TABLE 7
Comparison of nomogram results and other predictors

| Predictors | Accuracy (concordance index) |
|---|---|
| HGPIN history | 0.519 |
| ASAP history | 0.574 |
| PSA less than 4, 4-10, 10-20, | 0.638 |
| Greater than 20 ng/ml | |
| PSA slope greater than 0.75 ng/ml/yr | 0.615 |
| Our nomogram | 0.706 |

More recently, prostate volume has been described as a useful indicator for predicting positive repeat biopsy. Remzi et al. (2003) prospectively analyzed 861 patients with a PSA level of 4 to 10 ng/ml undergoing repeat biopsy 6 weeks after an initial negative biopsy. The total prostate volumes and transition zone volumes were measured. They found that using total prostate volume of less than 20 cm³ and greater than 80 cm³ as cutoffs would spare 7.1% of repeat biopsies. In addition, using transition zone volume cutoffs of less than 9 cm³ and greater than 41 cm³ would spare 10% of repeat biopsies.

Some variables described above, such as PSA density and percent free PSA, as well as race, prostate volume, and biopsy strategy, were not included in the nomogram as data was missing from many of the patients in the original group. As a result, these parameters were not recorded in the validation paper. However, those parameters may be included with, or replace one or more of, the parameters described above for a more accurate model. Even though using a heterogeneous population provides a more demanding test, this validation is not definitive and further validation with a prospective dataset still needs to be done.

Predicting whether a patient requires a repeat biopsy cannot be based on one factor alone. The nomogram is intended to be used as part of an approach that incorporates predicted probabilities, patient preferences, and clinical judgment.

### REFERENCES

American Cancer Society: Cancer Facts & Figures 2004, Atlanta, Ga., American Cancer Society (2004).
Breiman et al., Classification and Regression Trees. Monterey, Calif., Wadsworth and Brooks/Cole (1984).
Carlson et al., Urology, 52:455 (1998).
Cheville et al., Am. J. Clin. Pathol., 108:633 (1997).
Cox, J. Royal Statistical Society, B34:187 (1972)
Cristianni et al., An Introduction to Support Vector Machines, Cambridge University Press (2000).
Djavan et al., J. Clin. Oncol., 20:921 (2002).
Djavan et al., J. Urol., 163:1144 (2000).
Djavan et al., Urology, 54:517 (1999).
Eastham et al., Urology, 54:709 (1999).
Ellis et al., J. Urol., 153:1496 (1995).
Epstein et al., J. Urol., 160:2407 (1998).
Epstein et al., J. Urol., 160:97 (1998).
Epstein et al., J. Urol., 166:402 (2001).
Epstein et al., Urology, 162:1649 (1999).
Epstein, Am. J. Surg. Pathol., 22:1430 (1998).
Fleshner et al., J. Urol., 158:505 (1997).
Fowler et al., J. Urol., 163:813 (2000).
Hastie, The Elements of Statistical Learning, Springer (2001).
Hayek et al., Curr. Opin. Urol., 9:371 (1999).
Jemal et al., CA Cancer J. Clin., 52:23 (2002).
Keetch et al., J. Urol., 151:1571 (1994).
Keetch et al., J. Urol., 154:347 (1995).
Keetch et al., J. Urol., 156:428 (1996).
Lopez-Corona et al., J. Urol., 170:1184 (2003).
O'Dowd et al., Urology, 55:553 (2000).
Ouyang et al., Br. J. Urol., 87:70 (2001).
Park et al., J. Urol., 165:1409 (2001).
Perrotti et al., Urology, 162:1314 (1999).
Raviv et al., J. Urol., 156:1050 (1996).
Remzi et al., Urol., 61:161 (2003).
Roehl et al., Urology, 167:2435 (2002).
Roehrborn et al., Urology, 47:347 (1996).
Rumelhart et al., (eds), Parallel Distributed Processing: Exploration in the Microstructure of Cognition Volume 1, Foundations Cambridge, Mass., The MIT Press (1986).
Vis et al., Cancer, 92:524 (2001).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to determine a probability of a positive repeat prostate biopsy after a negative biopsy in a patient suspected of having prostate cancer, comprising:
a) detecting or determining four or more patient factors including age, digital rectal examination (DRE) results, prostate specific antigen (PSA) level, PSA slope, number of negative cores, family history of prostate cancer, history of high grade prostatic intraepithelial neoplasia (HGPIN), history of atypical small acinar proliferation (ASAP), number of months since initial negative biopsy, and/or number of months from previous negative biopsy; and
b) correlating the two or more patient factors with the probability of a positive repeat prostate biopsy after a negative biopsy in the patient.

2. A method to determine whether or when to repeat a prostate biopsy after a negative biopsy in a patient suspected of having prostate cancer, comprising:
a) detecting or determining two or more patient factors including age, DRE results, PSA level, PSA slope, number of negative cores, family history of prostate cancer, history of HGPIN, history of ASAP, number of months since initial negative biopsy, and/or number of months from previous negative biopsy; and
b) correlating the two or more patient factors to the determination of whether or when to repeat a prostate biopsy in the patient.

3. A method to determine a probability of a positive repeat prostate biopsy after a negative biopsy in a patient suspected of having prostate cancer, comprising:
a) inputting test information from the patient to a data input means, wherein the information includes age, DRE, PSA, PSA slope, number of negative cores, family history of prostate cancer, history of HGPIN, history of ASAP, number of months since initial negative biopsy, and/or number of months from previous negative biopsy;
b) executing a software for analysis of the test information; and
c) analyzing the test information so as to provide the probability of a positive repeat prostate biopsy in the patient.

4. A method to determine whether or when to repeat a prostate biopsy after a negative biopsy in a patient suspected of having prostate cancer, comprising:
a) inputting test information from the patient to a data input means, wherein the information includes age, DRE, PSA, PSA slope, number of negative cores, family history of prostate cancer, history of HGPIN, history of ASAP, number of months since initial negative biopsy, and/or number of months from previous negative biopsy;
b) executing a software for analysis of the test information; and
c) analyzing the test information so as to determine whether or when to repeat a prostate biopsy in the patient.

5. A method to predict a probability of a positive repeat prostate biopsy after a negative biopsy in a patient suspected of having prostate cancer, comprising:
a) correlating two or more factors for a patient to a functional representation of two or more factors determined for each of a plurality of persons previously subjected to two or more prostate biopsies, at least the first of which was negative and one of which was positive, so as to yield a value for total points for the patient, which factors for each of the plurality of persons is correlated with a probability of a positive repeat prostate biopsy after a negative biopsy for each person in the plurality, wherein the two or more factors include age, DRE results, PSA level, PSA slope, number of negative cores, family history, history of ASAP, history of HGPIN, number of months since previous negative biopsy, and/or number of months from an initial negative biopsy, wherein the functional representation comprises a scale for at least two of age, DRE, PSA, PSA slope, number of negative cores, family history, history of HGPIN, history of ASAP, number of months since initial negative biopsy, and/or number of months from previous negative biopsy, a points scale, a total points scale, and a predictor scale, wherein the scales for age, DRE, PSA, PSA slope, number of negative cores, family history, history of HGPIN, history of ASAP, number of months since initial negative biopsy, and/or number of months from previous negative biopsy, each have values which can be correlated with values on the points scale, and wherein the total points scale has values which may be correlated with values on the predictor scale; and
b) correlating the value on the total points scale for the patient with a value on the predictor scale to predict the quantitative probability of a positive repeat prostate biopsy after a negative biopsy in the patient.

6. A method to determine whether or when to repeat a prostate biopsy after a negative biopsy in a patient suspected of having prostate cancer, comprising:
a) correlating two or more factors for a patient to a functional representation of two or more factors determined for each of a plurality of persons previously subjected to two or more prostate biopsies, at least the first of which was negative and one of which was positive, so as to yield a value for total points for the patient, which factors for each of the plurality of persons is correlated with whether or when to repeat a prostate biopsy after a negative biopsy for each person in the plurality, wherein the two or more factors include age, PSA level, PSA slope, number of negative cores, family history, DRE results, history of ASAP, history of HGPIN, number of months since previous negative biopsy, and/or number of months from initial negative biopsy, wherein the functional representation comprises a scale of at least two of age, DRE, PSA, PSA slope, number of negative cores, family history, history of HGPIN, history of ASAP, number of months since initial negative biopsy, and/or number of months from previous negative biopsy, a points scale, a total points scale, and a predictor scale, wherein the scales for age, DRE, PSA, PSA slope, number of negative cores, family history, history of HGPIN, history of ASAP, number of months since initial negative biopsy, and/or number of months from previous negative biopsy, each have values which can be correlated with values on the points scale, and wherein the total points scale has values which may be correlated with values on the predictor scale; and
b) correlating the value on the total points scale for the patient with a value on the predictor scale to determine whether or when to repeat a prostate biopsy after a negative biopsy in a patient suspected of having prostate cancer.

7. The method of claim 5 or 6 wherein the functional representation is a nomogram.

8. The method of claim 7 wherein the nomogram is generated with a covariate Cox regression model.

9. The method of claim 1, 2, 5 or 6 wherein the correlating is conducted by a computer.

10. A machine-readable medium having instructions thereon for causing a suitably configured information-handling system to perform the method of any one of claims 1 to 9.

11. An apparatus, comprising:
a data input means, for input of test information from a patient after a negative prostate biopsy which patient is suspected of having prostate cancer, information including two or more of age, DRE, PSA, PSA slope, number of negative cores, family history, history of HGPIN, history of ASAP, number of months since initial negative biopsy, and/or number of months from previous negative biopsy;
a processor, executing a software for analysis of age, DRE, PSA, PSA slope, number of negative cores, family history, history of HGPIN, history of ASAP, number of months since initial negative biopsy, and/or number of months from previous negative biopsy;
wherein the software analyzes the age, DRE, PSA, PSA slope, number of negative cores, family history, history of HGPIN, history of ASAP, number of months since initial negative biopsy, and/or number of months from previous negative biopsy in the patient, and provides a probability of a positive repeat prostate biopsy in the patient.

12. An apparatus, comprising:
a data input means, for input of test information from a patient after a negative prostate biopsy which patient is suspected of having prostate cancer comprising two or more of age, DRE, PSA, PSA slope, number of negative cores, family history, history of HGPIN, history of ASAP, number of months since initial negative biopsy, and/or the number months from previous negative biopsy;
a processor, executing a software for analysis of age, DRE, PSA, PSA slope, number of negative cores, family history, history of HGPIN, history of ASAP, the number of months since initial negative biopsy, and/or the number of months from previous negative biopsy;
wherein the software analyzes the age, DRE, PSA, PSA slope, family history, history of HGPIN, history of ASAP, the number of months since initial negative biopsy, and/or the number of months from previous negative biopsy, and provides a determination of whether or when to repeat a prostate biopsy in the patient.

13. The apparatus of claim 11 or 12 wherein the test information is input manually using the data input means.

14. The apparatus of claim 11 or 12 wherein the software constructs a database of the test information.

15. An apparatus to predict a probability of a positive repeat prostate biopsy after a negative biopsy in a patient suspected of having prostate cancer, which apparatus comprises:
a) a correlation of two or more factors for each of a plurality of persons subjected to at least two prostate biopsies, at least the first of which was negative and one of which was positive, with the probability of a positive repeat prostate biopsy after a negative biopsy for each person of the plurality of persons, wherein the two or more factors include age, DRE, PSA, PSA slope, number of negative cores, family history, history of HGPIN, history of ASAP, the number of months since initial negative biopsy, and/or the number of months from previous negative biopsy; and
b) a means for comparing an identical two or more factors determined from the patient to the correlation to predict the quantitative probability of a positive repeat prostate biopsy in the patient.

16. An apparatus to determine whether or when to repeat a prostate biopsy after a negative biopsy in a patient suspected of having prostate cancer, which apparatus comprises:
a) a correlation of two or more factors for each of a plurality of persons subjected to at least two prostate biopsies, at least the first of which was negative and one of which was positive, with whether a repeat prostate biopsy is indicated after a negative biopsy for each person of the plurality of persons, wherein the two or more factors comprise age, DRE, PSA, PSA slope, number of negative cores, family history, history of HGPIN, history of ASAP, the number months since initial negative biopsy, and/or number of months from previous negative biopsy; and
b) a means for comparing an identical two or more factors determined from the patient to the correlation to determine whether or when to repeat a prostate biopsy in the patient.

17. A nomogram for the graphic representation of a quantitative probability of a positive repeat prostate biopsy after a negative biopsy in a patient suspected of having prostate cancer, comprising: a plurality of scales and a solid support, the plurality of scales being disposed on the support and comprising a scale for two or more of age, DRE, PSA, PSA slope, number of negative cores, family history, history of HGPIN, history of ASAP, number of months since initial negative biopsy, and/or the number months from previous negative biopsy, a points scale, a total points scale and a predictor scale, wherein the scales for age, DRE, PSA, PSA slope, number of negative cores, family history, history of HGPIN, history of ASAP, number of months since initial negative biopsy, and/or number of months from previous negative biopsy, each has values on the scales, and wherein the scales for age, DRE, PSA, PSA slope, number of negative cores, family history, history of HGPIN, history of ASAP, number of months since initial negative biopsy, and/or number of months from previous negative biopsy, are disposed on the solid support with respect to the points scale so that each of the values on the age, DRE, PSA, PSA slope, number of negative cores, family history, history of HGPIN, history of ASAP, number of months since initial negative biopsy, and/or number of months from previous negative biopsy scale, can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's age, DRE, PSA, PSA slope, number of negative cores, family history, history of HGPIN, history of ASAP, number of months since initial negative biopsy, and/or the number months from previous negative biopsy, can be added together to yield a total points value, and the total points value can be correlated with the predictor scale to predict a quantitative probability in the patient of a positive repeat prostate biopsy.

18. A nomogram for the graphic representation of the determination of whether or when to repeat a prostate biopsy after a negative biopsy in a patient suspected of having prostate cancer, comprising: a plurality of scales and a solid support, the plurality of scales being disposed on the support and comprising a scale for two or more of age, DRE, PSA, PSA slope, number of negative cores, family history, history of HGPIN, history of ASAP, number of months since initial negative biopsy, and/or the number months from previous negative biopsy, a points scale, a total points scale and a predictor scale, wherein the scales for age, DRE, PSA, PSA slope, number of negative cores, family history, history of HGPIN, history of ASAP, the number of months since initial negative biopsy, and/or number of months from previous negative biopsy, each has values on the scales, and wherein the scales for age, DRE, PSA, PSA slope, number of negative cores, family history, history of HGPIN, history of ASAP, number of months since initial negative biopsy, and/or number of months from previous negative biopsy, are disposed on the solid support with respect to the points scale so that each of the values on the age, DRE, PSA, PSA slope, number of negative cores, family history, history of HGPIN, history of ASAP, number of months since initial negative biopsy, and/or number of months from previous negative biopsy scale, can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's age, DRE, PSA, PSA slope, number of negative cores, family history, history of HGPIN, history of ASAP, number of months since initial negative biopsy, and/or the number months from previous negative biopsy, can be added together to yield a total points value, and the total points value can be correlated with the predictor scale to determine whether or when to repeat a prostate biopsy in the patient.

19. The nomogram of claim 17 or 18 wherein the solid support is a laminated card.

20. A method to predict a positive repeat prostate biopsy after a negative biopsy a patient suspected of having prostate cancer, comprising: determining two or more factors for a patient, which two or more factors include age, DRE, PSA, PSA slope, number of negative cores, family history, history of HGPIN, history of ASAP, number of months since initial negative biopsy, and/or the number months from previous negative biopsy; matching the factors to the values on the scales of the nomogram of claim 17; determining a separate point value for each of the factors; adding the separate point values together to yield a total points value; and correlating the total points value with a value on the predictor scale of the nomogram to predict a positive repeat prostate biopsy in the patient.

21. A method to determine whether or when to repeat a prostate biopsy after a negative biopsy a patient suspected of having prostate cancer, comprising: determining two or more factors for a patient, which two or more factors include age, DRE, PSA, PSA slope, number of negative cores, family history, history of HGPIN, history of ASAP, number of months since initial negative biopsy, and/or the number months from previous negative biopsy; matching the factors to the values on the scales of the nomogram of claim 18; determining a separate point value for each of the factors; adding the separate point values together to yield a total points value; and correlating the total points value with a value on the predictor scale of the nomogram to determine whether or when to repeat a prostate biopsy in the patient.

22. An apparatus for predicting a probability of a positive repeat prostate biopsy after a negative biopsy in a patient suspected of having prostate cancer, which apparatus comprises: a scale for two or more of age, DRE, PSA, PSA slope, number of negative cores, family history, history of HGPIN, history of ASAP, number of months since initial negative biopsy, and/or number of months from previous negative biopsy, a points scale, a total points scale and a predictor scale, wherein the scales for two or more of age, DRE, PSA, PSA slope, number of negative cores, family history, history of HGPIN, history of ASAP, number of months since initial negative biopsy, and/or number months from previous negative biopsy, each has values on the scales, and wherein the scales for age, DRE, PSA, PSA slope, number of negative cores, family history, history of HGPIN, history of ASAP, number of months since initial negative biopsy, and/or number of months from previous negative biopsy, are disposed so that each of the values on the age, DRE, PSA, PSA slope, number of negative cores, family history, history of HGPIN, history of ASAP, number of months since initial negative biopsy, and/or number of months from previous negative biopsy, can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's age, DRE, PSA, PSA slope, number of negative cores, family history, history of HGPIN, history of ASAP, number of months since initial negative biopsy, and/or number of months from previous negative biopsy, can be added together to yield a total points value, and the total points value can be correlated with the predictor scale to predict a positive repeat prostate biopsy in the patient.

23. An apparatus for predicting whether or when to repeat a prostate biopsy after a negative biopsy in a patient suspected of having prostate cancer, which apparatus comprises: a scale for two or more of age, DRE, PSA, PSA slope, number of negative cores, family history, history of HGPIN, history of ASAP, number of months since initial negative biopsy, and/or number of months from previous negative biopsy, a points scale, a total points scale and a predictor scale, wherein the scales for two or more of age, DRE, PSA, PSA slope, number of negative cores, family history, history of HGPIN, history of ASAP, number of months since initial negative biopsy, and/or number months from previous negative biopsy, each has values on the scales, and wherein the scales for age, DRE, PSA, PSA slope, number of negative cores, family history, history of HGPIN, history of ASAP, number of months since initial negative biopsy, and/or number of months from previous negative biopsy, are disposed so that each of the values on the age, DRE, PSA, PSA slope, number of negative cores, family history, history of HGPIN, history of ASAP, number of months since initial negative biopsy, and/or number of months from previous negative biopsy, can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's age, DRE, PSA, PSA slope, number of negative cores, family history, history of HGPIN, history of ASAP, number of months since initial negative biopsy, and/or number of months from previous negative biopsy, can be added together to yield a total points value, and the total points value can be correlated with the predictor scale to predict whether or when to repeat a prostate biopsy in the patient.

24. A system comprising:
a processor;
an input device;
an output device;
a storage device;
a database wherein the database includes data collected from a plurality of patients having more than one prostate biopsy, at least the first of which was negative and one of which was positive;
software operable on the processor to:
receive input from the input device, the input including two or more factors for determining a probability of a positive repeat prostate biopsy after a negative biopsy in a patient suspected of having prostate cancer; and
correlate received input with the collected data from the plurality of patients to determine a prognosis probability.

25. A system comprising:
a processor;
an input device;
an output device;
a storage device;
a database wherein the database includes data collected from a plurality of patients having more than one prostate biopsy, at least the first of which was negative and one of which was positive;
software operable on the processor to:
receive input from the input device, the input including two or more factors for determining whether or when to repeat a prostate biopsy after a negative biopsy in a patient suspected of having prostate cancer; and
correlate received input with the collected data from the plurality of patients to determine whether or when to perform a repeat biopsy in the patient.

26. The system of claim 24 or 25 wherein the software further includes a neural network model for correlating input data with the collected data from the plurality of patients having more than one prostate biopsy.

27. The system of claim 24 or 25 wherein the neural network model is a non-linear, feed-forward system of layered neurons which back-propagate prediction errors.

28. The system of claim 24 or 25 wherein the software further includes a recursive partitioning model for correlating input data with the collected data from the plurality of patients having more than one prostate biopsy.

29. The system of claim 24 or 25 wherein the software further includes vector machine technology for correlating input data with the collected data from the plurality of patients having more than one prostate biopsy.

30. The system of claim 24 or 25 further comprising a network connection.

31. The system of claim 24 or 25 wherein the network is the internet.

32. The system of claim 24 or 25 wherein the database is a relational database management system.

33. The system of claim 24 or 25 wherein the output device is a video display.

34. The system of claim 24 or 25 wherein the output device is a printer.

35. The system of claim 24 or 25 wherein the system is a personal computer.

36. The system of claim 24 or 25 wherein the system is a handheld computing device.

37. The system of claim 24 or 25 wherein the handheld computing device includes PalmOS.

38. The system of claim 24 or 25 wherein the database is accessible via the network.

39. The system of claim 24 or 25 wherein the system accepts input and provides output over the internet.

40. The system of claim 39 wherein the input is received and the output is provided in a markup language.

41. The system of claim 40 wherein the markup language is HTML.

42. A system for predicting a positive repeat prostate biopsy after a negative biopsy in a patient suspected of having prostate cancer, the system comprising:
a data structure for storing historic prostate cancer data, the structure contained in a memory and comprising two or more factors each corresponding to a characteristic of prostate cancer; and
a processing device including program means for correlating the two or more factors corresponding to characteristics of prostate cancer with factor data collected from a patient suspected of having prostate cancer, which patient has a negative prostate biopsy, wherein the correlating results in a probability of a positive repeat prostate biopsy output by the processing device.

43. A system for predicting whether or when to repeat prostate biopsy after a negative biopsy in a patient suspected of having prostate cancer, the system comprising:
a data structure for storing historic prostate cancer data, the structure contained in a memory and comprising two or more factors each corresponding to a characteristic of prostate cancer; and
a processing device including program means for correlating the two or more factors corresponding to characteristics of prostate cancer with factor data collected from a patient suspected of having prostate cancer, which patient has a negative prostate biopsy, wherein the correlating results in a probability of prostate cancer which is indicative of whether or when to repeat a prostate biopsy, output by the processing device.

44. The system of claim 42 or 43 wherein the factors include age, DRE, PSA, PSA slope, number of negative cores, family history, history of HGPIN, history of ASAP, months since initial negative biopsy, and/or months from previous negative biopsy.

45. A method for operating an information-processing device comprising:
maintaining a database of historic data wherein the historic data includes two or more scored factors corresponding to a plurality of patients having more than one prostate biopsy, at least the first of which was negative and one of which was positive;
collecting scores from a current patient with a negative biopsy for the two or more factors; and
correlating the scores collected from the current patient with the historic data to determine a probability of a positive repeat prostate biopsy in the current patient.

46. A method for operating an information-processing device comprising:
maintaining a database of historic data wherein the historic data includes two or more scored factors corresponding to a plurality of patients having more than one prostate biopsy, at least the first of which was negative and one of which was positive;
collecting scores from a current patient for the two or more factors; and
correlating the scores collected from the current patient with the historic data to determine a probability of prostate cancer which is indicative of whether or when to repeat a prostate biopsy in the patient.
